(19) United States
(12) Patent Application Publication
(10) Pub. No.: US 2008/0058557 A1
(43) Pub. Date: Mar. 6, 2008

(54) PREPARATION OF HIGH ASSAY DECABROMODIPHENYL OXIDE

(75) Inventor: Saadat Hussain, Baton Rouge, LA (US)

Correspondence Address: ALBEMARLE CORPORATION, 451 FLORIDA STREET, BATON ROUGE, LA, 70801-1765, US

(73) Assignee: ALBEMARLE CORPORATION, Baton Rouge, LA (US)

(21) Appl. No.: 11/843,742

(22) Filed: Aug. 23, 2007

Related U.S. Application Data
(60) Provisional application No. 60/823,824, filed on Aug. 29, 2006.

(51) Int. Cl. C07C 41/22

(52) U.S. Cl. 568/639

(57) ABSTRACT

This invention provides a process of preparing reaction-derived decabromodiphenyl oxide product of high purity. The process comprises feeding a solution comprising a solvent and diphenyl oxide and/or partially brominated diphenyl oxide into a reaction zone containing a refluxing reaction mixture comprising an excess of bromine and a catalytic amount of Lewis acid bromination catalyst. Substantially concurrently with the feeding, a sufficient amount of hydrogen bromide coproduct is removed from the reaction zone so as to form a reaction-derived decabromodiphenyl oxide product of high purity.

### REFERENCE TO RELATED APPLICATION

This application claims the benefit and priority of U.S. Provisional Application No. 60/823,824, filed Aug. 29, 2006, the disclosure of which is incorporated herein by reference.

### TECHNICAL FIELD

This invention relates to the preparation of high assay decabromodiphenyl oxide products.

### BACKGROUND

Decabromodiphenyl oxide (DBDPO) is a time-proven flame retardant for use in many flammable macromolecular materials, e.g., thermoplastics, thermosets, cellulosic materials, and back coating applications.

DBDPO is presently sold as a powder derived from the bromination of diphenyl oxide or a partially brominated diphenyl oxide containing an average of about 0.7 bromine atom per molecule of diphenyl oxide. Such bromination is conducted in excess bromine and in the presence of a bromination catalyst, usually AlCl₃. The operation is typically conducted at 177° F. (ca. 80.5° C.) with a 2 to 3 hour feed time. The powdered products are not 100% DBDPO, but rather are mixtures that contain up to about 98% DBDPO and about 1.5%, or a little more, of nonabromodiphenyl oxide co-product. As a partially brominated product, this amount of nonabromodiphenyl oxide is considered problematic by some environmental entities.

Bromination of diphenyl oxide and/or partially brominated diphenyl oxide is known in the art. See in this connection McKinnie, et al., U.S. Pat. No. 4,778,933. McKinnie, et al. discloses the production of a product having a DBDPO purity of 98.5 area % by gas chromatography analysis. While this teaching was a significant advance in the art, the process as taught required the use of a feed of molten diphenyl oxide, the handling of which requires slightly heating the feed during the process and can present certain challenges for commercial scale production.

It would therefore be desirable to provide process technology which does not depend upon the use of molten diphenyl oxide starting material while still enabling preparation of DBDPO products of purity at least as high as in McKinnie, et al. and higher than the known commercially available products, such as products comprising (i) at least 98.5% of DBDPO and (ii) nonabromodiphenyl oxide of less than 1.5%.

### SUMMARY OF INVENTION

It has now been found possible to directly produce, without recourse to recrystallization or chromatographic purification steps or molten feeds, DBDPO products having such higher amounts of DBDPO and lower contents of nonabromodiphenyl oxides. This can be accomplished by maintaining a substantially continuous, coordinated time-temperature feed of a solvent solution comprising diphenyl oxide (DPO) and/or partially brominated DPO to a reaction zone containing a refluxing reaction mixture comprising an excess of bromine and a catalytic amount of Lewis acid bromination catalyst, and substantially concurrently removing a sufficient amount of hydrogen bromide coproduct from the reaction zone so as to form a reaction-derived decabromodiphenyl oxide product of high purity.

One of the postulations upon which this invention was based is that when brominating DPO and/or partially brominated DPO with excess bromine in the presence of a Lewis acid catalyst, an equilibrium exists between nonabromodiphenyl oxide and decabromodiphenyl oxide which can be depicted as follows:
Br₉−DPO+Br₂⇄Br₁₀−DPO+HBr
and that a prolonged feed of the DPO and/or partially brominated DPO to refluxing bromine while substantially concurrently reducing hydrogen bromide content in the reactor enables a shift to the right in this equilibrium so that the amount of nonabromodiphenyl oxide is diminished and more of the desired decabromodiphenyl oxide forms and precipitates with less nonabromodiphenyl oxide being coprecipitated within the decabromodiphenyl oxide particles. It is further believed that the inclusion of sufficient solvent having a boiling point higher than bromine, such as methylene bromide or ethylene dibromide, in the DPO and/or partially brominated DPO feed enables the reaction temperature to increase without resorting to additional pressure, thereby promoting perbromination of the DPO/partially brominated DPO while in turn facilitating further removal of hydrogen bromide from the reaction mixture.

Thus, in accordance with one of the embodiments of this invention, there is provided a process of preparing reaction-derived decabromodiphenyl oxide of high purity, which process comprises feeding a solvent solution containing diphenyl oxide and/or partially brominated diphenyl oxide substantially continuously over a period in the range of about 1 to about 12 hours into a reactor containing a refluxing reaction mixture comprising an excess of bromine and a catalytic amount of Lewis acid bromination catalyst, and substantially concurrently removing a sufficient amount of hydrogen bromide coproduct from the reaction zone so as to form a reaction-derived decabromodiphenyl oxide product of high purity. In general, the duration of the feeding period is inversely related to the temperature at which the refluxing is occurring. In other words, the higher the temperature, the shorter can be the feed time.

As used herein including the claims:

1) The term “reaction-derived” means that the composition of the product is reaction determined and not the result of use of downstream purification techniques, such as recrystallization or chromatography, or like procedures that can affect the chemical composition of the product. Adding water or an aqueous base such as sodium hydroxide to the reaction mixture to inactivate the catalyst, and washing away of non-chemically bound impurities by use of aqueous washes such as with water or dilute aqueous bases are not excluded by the term “reaction-derived”. In other words, the products are directly produced in the synthesis process without use of any subsequent procedure to remove or that removes nonabromodiphenyl oxide from decabromodiphenyl oxide.

2) The term “high purity” means that the reaction-derived DBDPO product comprises at least 98.5% of DBDPO and nonabromodiphenyl oxide in an amount of less than 1.5% with, if any, a trace of octabromodiphenyl oxide. Preferably the process forms a reaction-derived product which comprises (i) at least 98.8% of DBDPO and (ii) nonabromodiphenyl oxide in an amount not exceeding 1.2%.

3) The term “substantially continuously” as regards the feeding means that the feeding is either totally continuous with no interruptions whatever or the feeding is interrupted one or more times as long as such interruptions are of short enough duration as not to affect in any significant way the end result of producing a reaction-derived product of high purity.

4) The term “substantially concurrently removing” as regards the amount of hydrogen bromide means that the removing is taking place at exactly the same time or at substantially the same time that the feeding is taking place. It is to be clearly understood that the feeding and the removing of the amount of hydrogen bromide need not start at the same moment in time as there can be a time lag between the commencement of the feed and the evolution of enough hydrogen bromide to initiate the removing of the amount thereof in the reactor. Likewise, if and when the feeding is terminated, there can be a period of time thereafter during which hydrogen bromide in the reactor can be removed. In addition, it should be understood that the term “substantially concurrently removing” includes one or more interruptions in the removal of hydrogen bromide as long as such interruptions are of short enough duration as not to affect in any significant way the end result of producing a reaction-derived product of high purity.

For the purposes of this invention, unless otherwise indicated, the % values given for DBDPO and nonabromodiphenyl oxide are to be understood as being the area % values that are derived from gas chromatography analysis. A recommended procedure for conducting such analyses is presented hereinafter.

Another embodiment is a process of preparing reaction-derived decabromodiphenyl oxide of high purity, which process comprises maintaining a substantially continuous, inversely related time-temperature feed of a solvent solution comprising diphenyl oxide (DPO) and/or partially brominated DPO to a reactor containing a refluxing reaction mixture comprising an excess of bromine and a catalytic amount of Lewis acid bromination catalyst, and substantially concurrently removing a sufficient amount of hydrogen bromide coproduct from the reaction zone so as to form a reaction-derived decabromodiphenyl oxide product of high purity. In this embodiment the higher the bromination reaction temperature, the shorter is the time duration of the feed, and the lower is the pressure in the reactor.

These and other embodiments and features of this invention will be still further apparent from the ensuing description, accompanying drawings and appended claims.

### BRIEF DESCRIPTION OF THE DRAWINGS

FIG. 1 is a copy of the GC trace of the product formed in Example 1 hereinafter.

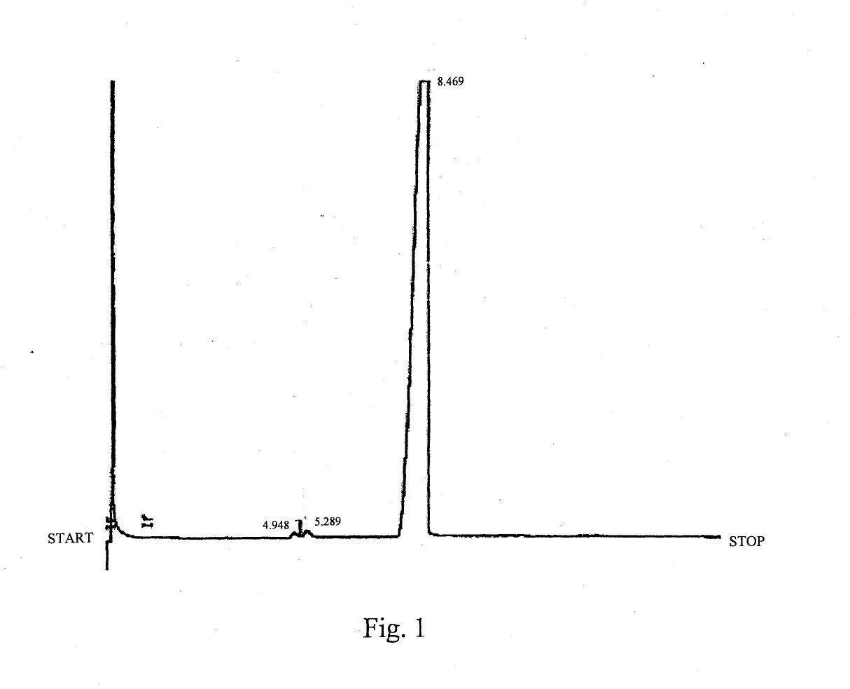

FIG. 2 is a copy of the GC trace of the product formed in Example 2 hereinafter.

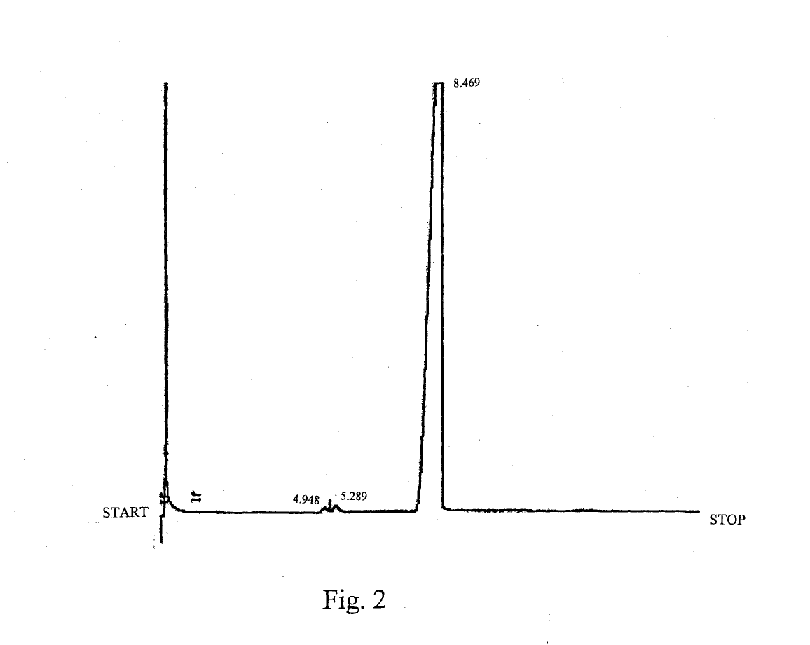

### FURTHER DETAILED DESCRIPTION OF THE INVENTION

This invention enables the preparation of highly pure DBDPO products that are derived from diphenyl oxide, one or more partially brominated diphenyl oxides, or any combination thereof. Such highly pure DBDPO products can be said to be “reaction-derived” since they are reaction determined and not the result of use of downstream purification techniques, such as recrystallization, chromatography, or like procedures. In other words, products of such high purity are directly produced in the synthesis process apart from use of subsequent purification procedures as applied to the recovered or isolated products. When decabromodiphenyl oxide having about 2% or more nonabromodiphenyl oxide is used pursuant to this invention, the processes of this invention can be viewed as a purification process.

The liquid reaction mixture is normally a liquid phase, with a small amount of solids formation as nonabromodiphenyl oxide and/or decabromodiphenyl oxide precipitate. In the practice of this invention, agitation of the liquid reaction mixture is advantageous.

In the practice of this invention, the solvent solution of DPO and/or partially brominated DPO is fed in the liquid state to the reaction mixture. The solvent solution may be fed to the reaction mixture above the surface, at the surface, or below the surface of the reaction mixture. It is preferred to feed the solvent solution subsurface to the reaction mixture.

It is to be noted that when the term “subsurface” is used anywhere in this document, including the claims, the term does not denote that there must be a headspace above the liquid mixture. For example, if the liquid mixture completely fills a reactor (with equal rates of incoming and outgoing flows), the term “subsurface” means in this case that the substance being fed subsurface is being fed directly into the body of the liquid mixture, the surface thereof being defined by the enclosing walls of the reactor.

It is recommended and preferred that the feed of solvent solution of DPO and/or partially brominated DPO commence at or after refluxing of the reaction mixture begins. The point at which decabromodiphenyl oxide has formed can be determined analytically by gas chromatography. Of course, the feed of diphenyl oxide and/or partially brominated diphenyl oxide need not begin at the very instant that refluxing begins; some delay in the initiation of the feed is acceptable.

The feed may be conducted at atmospheric, subatmospheric, or superatmospheric pressure. Operation at atmospheric or superatmospheric pressure is preferred; more preferable is operation at atmospheric pressure. It is alternatively preferred that the feed and distillation be conducted at autogenous pressure for a given set of reaction conditions. The temperature required to effect the distillation of HBr will vary with the pressure and the concentrations of HBr and brominated and unbrominated diphenyl oxide species present in the liquid mixture. As is well known in the art, the boiling point of bromine at atmospheric or slightly elevated pressures is about 59° C. Those skilled in the art will recognize that elevated pressures allow the attainment of higher temperatures for the distillation. However, one of the important features of the present invention is that the use of a sufficient solvent in appropriate amount in the process enables the elevation of temperature without resorting to pressure elevation, thereby enhancing the reaction without inhibiting the removal of hydrogen bromide from the reaction zone. The solvent used in processes of the invention may be a large number of candidate solvents, including for example, halogen-containing organic solvents such as, e.g., bromoform, methylene bromide, ethylene dibromide, n-propyl bromide, or the like, as well as, e.g., similar chlorine-based organic solvents. A particularly useful solvent is methylene bromide. The amount of the solvent may vary depending upon the solvent employed, but should be an amount sufficient to form a solution with the DPO and/or partially brominated DPO and to maintain or increase the reaction temperature to thereby enhance the removal of HBr from the reaction zone.

One consideration in the operation of the processes of this invention is the moderately low solubility of nonabromodiphenyl oxide and decabromodiphenyl oxide in bromine. Thus, it is desirable to keep enough bromine in the liquid mixture to prevent an acceleration of the precipitation of nonabromodiphenyl oxide and/or decabromodiphenyl oxide by adjusting the rate of the DPO and/or partially brominated DPO feed. In particularly preferred embodiments, the rate is adjusted so that the volume of the liquid mixture is constant or substantially constant.

In the processes of this invention, a flow of inert gas can be passed through the reaction zone during the feed and/or upon completion of the feed. Examples of suitable inert gases include nitrogen, helium, and argon; nitrogen is a preferred inert gas.

Another feature of this invention is that, once separated from the liquid mixture, the HBr in the distillate may be oxidized to form bromine, for example by treatment with hydrogen peroxide. Alternatively, the HBr may be separated from the bromine and used or sold.

Termination of the bromination reaction is typically effected by deactivating the catalyst with water and/or an aqueous base such as a solution of sodium hydroxide or potassium hydroxide.

The Lewis acid catalyst in the liquid mixture can be any of various iron and/or aluminum Lewis acids. These include the metals themselves such as iron powder, aluminum foil, or aluminum powder, or mixtures thereof. Preferably use is made of such catalyst materials as, for example, ferric chloride, ferric bromide, aluminum chloride, aluminum bromide, or mixtures of two or more such materials. More preferred are aluminum chloride and aluminum bromide with addition of aluminum chloride being more preferred from an economic standpoint. It is possible that the makeup of the catalyst may change when contained in the liquid mixture. The Lewis acid should be employed in an amount sufficient to effect a catalytic effect upon the bromination reaction being conducted. Typically, the amount of Lewis acid used will be in the range of about 0.06 to about 2 wt %, and preferably in the range of about 0.2 to about 0.7 wt % based on the weight of the bromine being used.

In the various embodiments of this invention, the diphenyl oxide species can be diphenyl oxide (DPO) itself, one or more partially brominated diphenyl oxides, or a mixture of DPO and one or more partially brominated diphenyl oxides.

Partially brominated DPO, which can be used in the practice of this invention, typically contains in the range of about 0.5 to about 4 atom(s) of bromine per molecule of DPO. Partially brominated diphenyl oxides with more than about 4 atoms of bromine per molecule can be used in the processes of this invention. The processes of this invention can be applied to a decabromodiphenyl oxide product that contains about 2% or more nonabromodiphenyl oxide. Decabromodiphenyl oxide that is currently commercially available typically contains about 2% nonabromodiphenyl oxide.

The DBDPO products formed in processes of this invention are white or slightly off-white in color. White color is advantageous as it simplifies the end-user's task of insuring consistency of color in the articles that are flame retarded with the DBDPO products.

The processes of this invention can produce high purity decabromodiphenyl oxide products which comprise (i) at least 98.5% of DBDPO and (ii) nonabromodiphenyl oxide of less than 1.5%. In some instances, the processes of this invention can produce high purity decabromodiphenyl oxide products which comprise (i) at least 98.8% decabromodiphenyl oxide and (ii) nonabromodiphenyl oxide in an amount not exceeding 1.2%.

The DBDPO products formed in the processes of this invention may be used as flame retardants in formulations with virtually any flammable material. The material may be macromolecular, for example, a cellulosic material or a polymer. Illustrative polymers are: olefin polymers, cross-linked and otherwise, for example homopolymers of ethylene, propylene, and butylene; copolymers of two or more of such alkene monomers and copolymers of one or more of such alkene monomers and other copolymerizable monomers, for example, ethylene/propylene copolymers, ethylene/ethyl acrylate copolymers and ethylene/propylene copolymers, ethylene/acrylate copolymers and ethylene/vinyl acetate copolymers; polymers of olefinically unsaturated monomers, for example, polystyrene, e.g. high impact polystyrene, and styrene copolymers, polyurethanes; polyamides; polyimides; polycarbonates; polyethers; acrylic resins; polyesters, especially poly(ethyleneterephthalate) and poly(butyleneterephthalate); polyvinyl chloride; thermosets, for example, epoxy resins; elastomers, for example, butadiene/styrene copolymers and butadiene/acrylonitrile copolymers; terpolymers of acrylonitrile, butadiene and styrene; natural rubber; butyl rubber and polysiloxanes. The polymer may be, where appropriate, cross-linked by chemical means or by irradiation. The DBDPO products of this invention can be used in textile applications, such as in latex-based back coatings.

The amount of a DBDPO product of this invention used in a formulation will be that quantity needed to obtain the flame retardancy sought. It will be apparent to those skilled in the art that for all cases no single precise value for the proportion of the product in the formulation can be given, since this proportion will vary with the particular flammable material, the presence of other additives and the degree of flame retardancy sought in any give application. Further, the proportion necessary to achieve a given flame retardancy in a particular formulation will depend upon the shape of the article into which the formulation is to be made, for example, electrical insulation, tubing, electronic cabinets and film will each behave differently. In general, however, the formulation, and resultant product, may contain from about 1 to about 30 wt %, preferably from about 5 to about 25 wt % DBDPO product of this invention. Master batches of polymer containing DBDPO, which are blended with additional amounts of substrate polymer, typically contain even higher concentrations of DBDPO, e.g., up to 50 wt % or more.

It is advantageous to use the DBDPO products of this invention in combination with antimony-based synergists, e.g. Sb₂O₃. Such use is conventionally practiced in all DBDPO applications. Generally, the DBDPO products of this invention will be used with the antimony based synergists in a weight ratio ranging from about 1:1 to 7:1, and preferably of from about 2:1 to about 4:1.

Any of several conventional additives used in thermoplastic formulations may be used, in their respective conventional amounts, with the DBDPO products of this invention, e.g., plasticizers, antioxidants, fillers, pigments, UV stabilizers, etc.

Thermoplastic articles formed from formulations containing a thermoplastic polymer and DBDPO product of this invention can be produced conventionally, e.g., by injection molding, extrusion molding, compression molding, and the like. Blow molding may also be appropriate in certain cases.

Recommended Gas Chromatographic Procedure

The gas chromatography is on a Hewlett-Packard 5890, series II, with Hewlett-Packard model 3396 series II integrator, the software of which is that installed with the integrator by the manufacturer. The gas chromatograph column used is an aluminum clad fused silica column, Code 12 AQ5 HT5 (Serial number A132903) obtained from SGE Scientific, with film thickness of 0.15 micron. The program conditions are: initial start temperature 250° C., ramped up to 300° C. at a rate of 5° C./min. The column head pressure is 10 psig (ca. 1.70×10⁵ Pa). The carrier gas is helium. The injection port temperature is 275° C. and the flame ionization temperature is 325° C. Samples are prepared by dissolving ca. 0.1 g in 8-10 mL of dibromomethane. The injection size is 2.0 microliters.

The GC procedure described above provides a trace having a plurality of peaks. The first peak is deemed to be the meta- and para-hydrogen isomers of nonabromodiphenyl oxide. The second peak is deemed to be the ortho-hydrogen isomer of nonabromodiphenyl oxide. The main peak, of course, is decabromodiphenyl oxide.

The following examples are presented for purposes of illustration, and are not intended to impose limitations on the scope of this invention.

### EXAMPLE 1

A reactor was configured using a heating mantle, a 1-liter, 4 neck flask equipped with a mechanical stirrer, a thermometer, an addition funnel equipped with a 1/16 inch Teflon® dip tube, an ice cold caustic scrubber and a nitrogen gas inlet to sweep the reactor with nitrogen gas (N₂). The reactor was charged with 323 mL of bromine (Br₂) and 4.3 g of AlCl₃ catalyst. The resulting mixture was stirred and heated to a temperature of 55° C. The addition funnel was then charged with a solution of diphenyl oxide (42.5 g) in dibromomethane (75 mL), which solution was then added, sub-surface, to the bromine/AlCl₃ reactor mixture using the dip tube and with agitation of that reactor mixture. Addition was complete after 52 minutes, after which time the reactor mixture was stirred and heated to about 69° C. The scrubber weight gain observed was 255.6 g after the first hour and 10 minutes of refluxing at 69° C. After 2 hours and 10 minutes of refluxing at 69° C., an additional 50 mL of fresh dibromomethane was added to the reaction mixture to increase reflux temperature to 73° C., and the reflux at this temperature was continued for an additional 45 minutes, after which time the heat source was removed and the mixture was allowed to cool slowly. The total reflux time was 2 hours and 10 minutes at 69° C. followed by 1 hour and 45 minutes at 73° C. After cooling overnight, 300 mL of water was added to the reaction mixture, the reactor was set to stir the reaction mixture and distill therefrom the excess bromine. In fifteen minutes the reaction mixture reached 67° C. and distillation of bromine/dibromomethane/water began. Distilled bromine and dibromomethane were continuously removed and distilled water was recycled back to the reactor vessel during the next 25 minutes, at which time the mixture temperature was 69° C. and 100 mL of bromine/dibromomethane had been distilled. Distillation continued for another 1 hour and 20 minutes until substantially all bromine/dibromomethane was distilled. Heat was removed and the reactor content was allowed to cool to room temperature. Caustic then was added to the contents, with stirring, to a pH in the range of 12-13. The contents then was filtered and the resulting cake washed with water (6×150 mL) and allowed to dry in air overnight. The cake when wet weighed 246.3 g, and when dried in air overnight to shiny crystals, it weighed 233.9 g. GC analysis of those crystals revealed nonabromodiphenyl oxide (isomer 1) content of 0.50 area %, nonabromodiphenyl oxide (isomer 2) content of 0.65 area %, and decabromodiphenyl oxide content of 98.84 area %.

### EXAMPLE 2

Using equipment identical to that of Example 1, 42.5 g of diphenyl oxide was dissolved in 125 mL methylene bromide and charged to the additional funnel. The reactor was charged with bromine (226 mL Br₂) and catalyst (4.25 g AlCl₃). The reactor contents was stirred and heated to 60° C. Then addition of the additional funnel charge was initiated via the dip tube. After feeding the funnel contents over a period of 1 hour and 15 minutes, the temperature of the reactor was 63° C. Additional heat was applied to the reactor and 11 minutes later the temperature was 80° C. with the reaction mass refluxing. After 35 minutes of refluxing the temperature remained at 80° C. and the scrubber gain was 257.2 g (compared to theoretical gain of 2.5 moles HBr, 202.5 g). Reflux was continued for a total reflux time of 6 hours, whereupon heat source was cut and the reaction mass was allowed to cool. Agitation of the reaction mass was stopped 36 minutes after the heat source was cut, and the reactor content was allowed to stand overnight. The next morning, 350 mL of water was added to the reactor and the content was stirred well. Steam distillation of the reactor content was commenced with heating. Bromine and methylene bromide began to distill over at 71° C., and water was recycled back to the reactor with separation of the bromine and methylene bromide into a flask. After 1 hour and 10 minutes, the distillation was complete and the reactor temperature was 100° C. The heat source was cut and contents allowed to cool. Sodium hydroxide (aqueous) was added to a pH of 10-11 with stirring. Reactor content was filtered and the resulting cake was washed with water (4×150 mL). The wet cake weight was 253.0 g, and it was allowed to air dry overnight. The dried wet cake weight was 235.0 g (0.244 mole, 98%). GC analysis of the dried product revealed 98.54 area % DBDPO.

It is to be understood that the reactants and components referred to by chemical name or formula anywhere in this document, whether referred to in the singular or plural, are identified as they exist prior to coming into contact with another substance referred to by chemical name or chemical type (e.g., another reactant, a solvent, or etc.). It matters not what preliminary chemical changes, transformations and/or reactions, if any, take place in the resulting mixture or solution or reaction medium as such changes, transformations and/or reactions are the natural result of bringing the specified reactants and/or components together under the conditions called for pursuant to this disclosure. Thus the reactants and components are identified as ingredients to be brought together in connection with performing a desired chemical operation or reaction or in forming a mixture to be used in conducting a desired operation or reaction. Also, even though an embodiment may refer to substances, components and/or ingredients in the present tense (“is comprised of”, “comprises”, “is”, etc.), the reference is to the substance, component or ingredient as it existed at the time just before it was first contacted, blended or mixed with one or more other substances, components and/or ingredients in accordance with the present disclosure.

Also, even though the claims may refer to substances in the present tense (e.g., “comprises”, “is”, etc.), the reference is to the substance as it exists at the time just before it is first contacted, blended or mixed with one or more other substances in accordance with the present disclosure.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

Each and every patent or other publication or published document referred to in any portion of this specification is incorporated in toto into this disclosure by reference, as if fully set forth herein.

The foregoing description is made with reference to preferred embodiments and should not be construed as being limited to those illustrations. Rather, the invention is susceptible to considerable variation within the spirit and scope of the appended claims and such other claims as may be asserted on the basis of the foregoing description in accordance with applicable law.

1. A process of preparing reaction-derived decabromodiphenyl oxide product of high purity, which process comprises feeding a solution comprising a solvent and diphenyl oxide and/or partially brominated diphenyl oxide into a reaction zone containing a refluxing reaction mixture comprising an excess of bromine and a catalytic amount of Lewis acid bromination catalyst, and substantially concurrently removing a sufficient amount of hydrogen bromide coproduct from the reaction zone so as to form a reaction-derived decabromodiphenyl oxide product of high purity.

2. A process as in claim 1 wherein a flow of inert gas is passed through the reaction zone during the feed and/or upon completion of the feed.

3. A process as in claim 1 wherein the process is carried out under autogenous pressure.

4. A process as in claim 1 wherein the pressure is maintained at substantially atmospheric pressure.

5. A process as in claim 1 wherein the solvent is a halogen-containing organic solvent.

6. A process as in claim 5 wherein the solvent is a bromine-containing organic solvent.

7. A process as in claim 6 wherein the solvent is methylene bromide.

8. A process as in claim 1 wherein said high purity decabromodiphenyl oxide product contains (i) at least 98.5% decabromodiphenyl oxide and (ii) nonabromodiphenyl oxide in an amount not exceeding 1.5%.

9. A process of preparing reaction-derived decabromodiphenyl oxide of high purity, which process comprises maintaining a substantially continuous, inversely related time-temperature feed of a solvent solution comprising diphenyl oxide and/or partially brominated diphenyl oxide to a reactor containing a refluxing reaction mixture comprising an excess of bromine and a catalytic amount of Lewis acid bromination catalyst, and substantially concurrently removing a sufficient amount of hydrogen bromide coproduct from the reaction zone so as to form a reaction-derived decabromodiphenyl oxide product of high purity.
